(19) United States
(12) Patent Application Publication
(10) Pub. No.: US 2025/0276182 A1
(43) Pub. Date: Sep. 4, 2025

(54) SYSTEMS AND METHODS FOR ADAPTIVE VAGUS NERVE STIMULATION

(71) Applicant: The Alfred E. Mann Foundation for Scientific Research, Valenicia, CA (US)

(72) Inventor: John RIVERA, Oxnard, CA (US)

(21) Appl. No.: 19/067,773

(22) Filed: Feb. 28, 2025

Related U.S. Application Data
(60) Provisional application No. 63/559,641, filed on Feb. 29, 2024.

(51) Int. Cl. A61N 1/36; A61B 5/00; A61B 5/0205; A61B 5/021; A61B 5/024; A61B 5/08; A61B 5/145; A61B 5/28; A61B 5/291; A61B 5/296; A61B 5/363; A61N 1/05; G16H 50/30

(52) U.S. Cl. CPC A61N 1/36139; A61B 5/0205; A61B 5/021; A61B 5/024; A61B 5/0816; A61B 5/14542; A61B 5/28; A61B 5/291; A61B 5/296; A61B 5/363; A61B 5/4809; A61B 5/4818; A61B 5/4836; A61B 5/7264; A61N 1/0556; A61N 1/36053; A61N 1/3615; A61N 1/36171; A61N 1/36175; G16H 50/30

(57) ABSTRACT

The present disclosure relates to systems and methods for vagus nerve stimulation that utilize adaptive stimulation algorithms and sensor data collected from one or more external or implanted sensors.

### CROSS-REFERENCE TO RELATED APPLICATION

The present application claims the benefit of priority to U.S. Provisional Application No. 63/559,641, filed Feb. 29, 2024, the entire contents of which is incorporated by reference in its entirety.

### TECHNICAL FIELD

The present disclosure relates to systems and methods for vagus nerve stimulation that utilize adaptive stimulation algorithms.

### BACKGROUND

Epilepsy is a disorder of the brain characterized by repeated seizures. Epilepsy can be treated under appropriate circumstances with vagus nerve stimulation (“VNS”). VNS entails the surgical implantation of a stimulator device into a patient's chest area under the skin to stimulate the vagus nerve with electrical stimulus pulses. The vagus nerve originates from the brainstem and traverses both sides of the neck down to the chest and abdomen. The VNS device sends electrical signals via the vagus nerve to the brain. A lead wire having a cuff at the proximal end connects the stimulator device to the vagus nerve. The cuff has one or more electrodes within the cuff and, when implanted, encircles the vagus nerve. VNS has been shown to be helpful in many cases for reducing the number and severity of seizures, particularly for patients who are less responsive to more non-invasive methods like oral medication. VNS has also been shown to reduce depression in certain treatment-resistant patients.

VNS provides a variety of clinical benefits. However, VNS is known to result in an increase in respiratory rate, and a decrease in respiratory amplitude, tidal volume, and oxygen saturation during periods of stimulation. As such, most patients that utilize VNS experience an increase in their apnea-hypopnea index (“AHI”) after VNS implantation. This presents a concern for patients suffering from preexisting obstructive sleep apnea (“OSA”), in that apneas and hypopneas occur more frequently during VNS. Research has shown that approximately one-third of subjects afflicted with medically refractory seizures are also diagnosed with OSA. As such, a sizable population of subjects are affected by this issue.

Further research in this area has demonstrated that treating OSA also provides improved seizure control as an added benefit, with positive results observed in large population studies regardless of changes to antiepileptic medications. These respiratory events can be reduced with changes in the VNS operational parameters or with the use of a continuous positive airway pressure (“CPAP”) device. Current treatment options for subjects afflicted with OSA and in need of VNS are limited. Subjects can choose to test various combinations of VNS parameters in the hope of arriving at a successful result, utilize positive airway pressure therapy (e.g., with a CPAP device), or discontinue VNS entirely. In each case, the choice of therapy needs to be individualized and designed by a clinician, requiring a significant investment of time and medical resources.

### SUMMARY OF SELECTED ASPECTS

The devices, systems, and methods for adaptive VNS described herein address various shortcomings in the art, e.g., by utilizing adaptive algorithms to automatically select and optimize stimulation parameters based on sensor data collected from the subject. These devices, systems, and methods provide various advantages compared to prior solutions, e.g., they can be used to automatically optimize treatment parameters with limited need for input from a clinician. Moreover, personalized treatment parameters should improve treatment efficacy, and foster more widespread adoption of VNS by subjects that were previously unable to take advantage of VNS due to preexisting OSA concerns.

In a first general aspect, the disclosure provides a system for vagus nerve stimulation, comprising: a stimulator implanted in a subject and configured to deliver electrical stimulation to a vagus nerve of the subject and to a hypoglossal nerve of the subject; and one or more sensors, each configured to detect a signal indicative of a level of a physiological biomarker of the subject; and a controller configured to determine one or more OSA indicators (e.g., the subject's AHI, respiration rate, oxygen desaturation index (“ODI”), and/or any other marker(s) indicative of the subject experiencing an obstructive sleep apnea), based on at least one signal detected by the one or more sensors, and control delivery of the electrical stimulation to the vagus nerve of the subject, and delivery of the electrical stimulation to the hypoglossal nerve of the subject, based on the determined OSA indicator(s).

In some aspects, the controller is further configured to pause stimulation of the vagus nerve in response to determining an increase in the subject's OSA indicator(s), beyond at least one preset threshold. In some aspects, a preset threshold may comprise the subject having an AHI≥15 episodes, an ODI≥15 episodes, a respiratory rate <12 breaths per minute, or any combination thereof. In some aspects, a preset threshold may comprise the subject having an AHI≥10, 11, 12, 13, 14, or 15 episodes, an ODI≥10, 11, 12, 13, 14, or 15 episodes, a respiratory rate <10, 11, 12, 13, 14, or 15 breaths per minute, or any combination thereof.

In some aspects, the controller is further configured to initiate stimulation of the hypoglossal nerve of the subject in response to determining an increase in the subject's OSA indicator(s), beyond at least one preset threshold. This threshold may be the same as a threshold used for determining whether to pause stimulation of the vagus nerve (e.g., as described in the preceding passage) or an independently selected threshold. In some aspects, a preset threshold for determining whether to initiate stimulation of the hypoglossal nerve may comprise the subject having an AHI≥15 episodes, an ODI≥15 episodes, a respiratory rate <12 breaths per minute, or any combination thereof. In some aspects, a preset threshold may comprise the subject having an AHI≥10, 11, 12, 13, 14, or 15 episodes, an ODI≥10, 11, 12, 13, 14, or 15 episodes, a respiratory rate <10, 11, 12, 13, 14, or 15 breaths per minute, or any combination thereof.

In some aspects, the controller is further configured to pause stimulation of the vagus nerve and initiate stimulation to the hypoglossal nerve, in response to determining an increase in the subject's OSA indicator(s), beyond the at least one preset threshold.

In some aspects, the controller is further configured to resume administering stimulation to the vagus nerve after initiating stimulation to the hypoglossal nerve.

In some aspects, the controller is further configured to adjust one or more parameters of the electrical stimulation administered to the vagus nerve, prior to resuming the administration of stimulation to the vagus nerve. Adjustment may comprise, e.g., decreasing a pulse frequency and/or amplitude of stimulation. In some aspects, a pulse frequency of stimulation may be decreased (e.g., from 30 Hz to 15 Hz or below) and/or the pulse amplitude may be decreased to a level below 2 mA. In some aspects, the adjustment comprises decreasing a pulse frequency of stimulation by 1, 2, 3, 4, 5, 6, 7, 8, 9, 10, 11, 12, 13, 14, 15, 16, 17, 18, 19, or 20 Hz, or by an amount within a range defined by any pair of the foregoing values. In some aspects, the pulse amplitude may be decreased stepwise (e.g., by 1, 2, 3, 4, or 5 Hz). In some aspects, the adjustment comprises decreasing a pulse amplitude of stimulation by 0.1, 0.2, 0.3, 0.4, 0.5, 0.6, 0.7, 0.8, 0.9, 1.0 mA, or by an amount within a range defined by any pair of the foregoing values. In some aspects, the adjustment process may comprise multiple rounds of decreasing one or more stimulation parameters (e.g., until the subject's OSA indicator(s), are below the at least one preset threshold for pausing stimulation of the vagus nerve or initiating stimulation of the hypoglossal nerve).

In some aspects, the controller is further configured to disable hypoglossal nerve stimulation when (i) the subject's OSA indicator(s), exceeds the at least one preset threshold, and (ii) the subject is determined to be awake. In some aspects, a preset threshold for determining whether to disable hypoglossal nerve stimulation may comprise a threshold amount of movement or a change in posture of the subject (e.g., indicating that the subject is awake or likely awake).

In some aspects, the controller is further configured to adjust one or more parameters of the electrical stimulation administered to the vagus nerve after disabling the hypoglossal nerve stimulation. Adjustment may comprise, e.g., decreasing a pulse frequency and/or amplitude of stimulation. In some aspects, a pulse frequency of stimulation may be decreased (e.g., from 30 Hz to 15 Hz or below) and/or the pulse amplitude may be decreased to a level below 2 mA. In some aspects, the adjustment comprises decreasing a pulse frequency of stimulation by 1, 2, 3, 4, 5, 6, 7, 8, 9, 10, 11, 12, 13, 14, 15, 16, 17, 18, 19, or 20 Hz, or by an amount within a range defined by any pair of the foregoing values. In some aspects, the pulse amplitude may be decreased stepwise (e.g., by 1, 2, 3, 4, or 5 Hz). In some aspects, the adjustment comprises decreasing a pulse amplitude of stimulation by 0.1, 0.2, 0.3, 0.4, 0.5, 0.6, 0.7, 0.8, 0.9, 1.0 mA, or by an amount within a range defined by any pair of the foregoing values. In some aspects, the adjustment process may comprise multiple rounds of decreasing one or more stimulation parameters (e.g., until the subject's OSA indicator(s), are below the at least one preset threshold for pausing stimulation of the vagus nerve or initiating stimulation of the hypoglossal nerve).

In some aspects, the one or more parameters of the electrical stimulation comprise a pulse frequency, width, amplitude, and/or duty cycle of the electrical stimulation delivered to the vagus nerve.

In some aspects, the at least one preset threshold comprises: a threshold AHI, respiration rate, or ODI level selected by a clinician; a threshold AHI, respiration rate, or ODI level selected by a clinician based on historical AHI, respiration rate, or ODI level of the subject; and/or a threshold AHI, respiration rate, or ODI level previously determined to define a tolerable or intolerable AHI, respiration rate, or ODI level for the subject. Similar thresholds may be provided for any other selected OSA indicator(s).

In some aspects, the at least one preset threshold comprises a plurality of thresholds, optionally including a threshold for the AHI, a threshold for the respiration rate, and/or a threshold for the ODI; and wherein the controller is configured to administer or disable stimulation based on two or more of the plurality of thresholds. In other aspects, the plurality of thresholds may apply to any other selected OSA indicator(s).

In some aspects, the one or more sensors comprise a heart rate sensor, a blood pressure sensor, an SpO₂ sensor, an electroencephalogram (“EEG”) sensor, an electrocardiogram (“ECG”) sensor); and/or an electromyography (“EMG”) sensor.

In some aspects, the stimulator comprises a pulse generator configured to deliver electrical stimulation to the hypoglossal and vagus nerves of the subject. For example, a bifurcated electrode lead connected to a plurality of electrodes placed on a hypoglossal nerve cuff, and a plurality of electrodes placed on a vagus nerve cuff, may be used in some embodiments. In some aspects, the stimulator may utilize a lead adaptor to provide lead connections for separate nerve targets (e.g., the hypoglossal and vagus nerves).

In some aspects, the controller is further configured to detect the onset of a seizure in the subject, based on at least one signal detected by the one or more sensors, and to control the delivery of the electrical stimulation to the vagus nerve and/or to the hypoglossal nerve of the subject based on the detection of the seizure. In some aspects, the controller is further configured to control the delivery of the electrical stimulation to the vagus nerve and/or to the hypoglossal nerve of the subject based on manual input from the subject indicating the onset, or expected onset, of a seizure.

In some aspects, the controller is further configured to detect ictal tachycardia events experienced by the subject, based on at least one signal detected by the one or more sensors.

In some aspects, the controller is further configured to detect the onset of a seizure in the subject based on the detection of an ictal tachycardia event experienced by the subject.

In a second general aspect, the disclosure provides a method for VNS, comprising: a) providing a stimulator implanted in a subject and configured to deliver electrical stimulation to a vagus nerve of the subject, and to a hypoglossal nerve of the subject; b) detecting, by at least one sensor, a signal indicative of a level of a physiological biomarker of the subject; c) providing a controller configured to i) determine one or more OSA indicators for the subject (e.g., an AHI respiration rate ODI, or a combination thereof), based on at least one signal detected by the one or more sensors, and ii) control the delivery of the electrical stimulation to the vagus nerve of the subject, and/or to the hypoglossal nerve of the subject, based on the determined OSA indicator(s).

The methods for VNS disclosed herein may utilize any of the systems described herein and any components thereof, alone or in combination. For example, such methods may utilize systems configured according to any of the aspects described in this section.

### BRIEF DESCRIPTION OF THE DRAWINGS

FIG. 1 is a block diagram illustrating an exemplary embodiment of a system for adaptive VNS in accordance with the present disclosure. In this example, a single implanted housing (101) contains a stimulator (102), controller (103), and an optional sensor (106), wherein the housing (101) is further connected to two nerve cuffs (each comprising one or more electrodes) via a bifurcated electrode lead. The controller (103) in this VNS device is further shown to be in communication with various sensors (106) via a series of wireless or wired connections, and with a remote server (109).

FIG. 2 is a conceptual flow diagram of a process for treating a subject using VNS using an adaptive algorithm according to an exemplary aspect of the disclosure.

FIG. 3 is a diagram showing an IPG, a lead adaptor (in this case, a two-way splitter), and two stimulation leads having nerve cuffs on each lead.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (“GPUs”), central processing units (“CPUs”), application processors, digital signal processors (“DSPs”), reduced instruction set computing (“RISC”) processors, systems on a chip (“SoC”), baseband processors, field programmable gate arrays (“FPGAs”), programmable logic devices (“PLDs”), application-specific integrated circuits (“ASICs”), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (“RAM”), a read-only memory (“ROM”), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

As explained above, the systems and methods provided herein are designed to improve VNS efficacy by managing both seizure and AHI events in a personalized, adaptive manner. For example, in some aspects systems according to the disclosure may comprise an implanted stimulator (102) (e.g., contained within a housing (101) that also includes a controller (103), wherein the stimulator is connected to a bifurcated electrode lead whereby N electrodes are placed on a VNS cuff and M electrodes are placed on a hypoglossal nerve cuff (the N and M electrode counts being independently selected and each including 1, 2, 3, 4, 5, 6, 7, 8, 9, 10 or more electrodes). The electrodes can be hosted in varying configurations such as a cuff, cylindrical, paddle or any other nerve-accommodating configuration. The controller (103) may be configured to control the administration of electrical stimulation from the stimulator (102) to the vagus nerve and/or the hypoglossal nerve using an adaptive algorithm that takes into account sensor data from one or more sensors (106) communicatively linked to the controller (103). In some aspects, the controller (103) may, e.g., be configured to monitor ictal tachycardia events and/or one or more additional physiological biomarkers or activities of the subject being treated, in order to identify the onset of seizures experienced by the subject. The controller (103) may also, or alternatively, be configured to monitor the respiration rate and/or related physiological biomarkers of the subject (e.g., heart rate, oxygen saturation, “SpO₂” level) to determine or detect one or more OSA indicators (e.g., to determine an AHI and/or detect AHI events experienced by the subject). Upon detection of one or more OSA indicators exceeding a threshold or range (e.g., an elevated AHI, respiration rate, ODI, or a combination of one ort more of these indicators) during VNS, the controller (103) may be configured to respond by adjusting one or more parameters of the VNS until airway patency and/or the AHI is restored to pre-VNS levels.

As used herein, the term “apnea-hypopnea index,” or “AHI,” refers to the metric for evaluating the severity of OSA. The AHI is calculated by dividing the number of episodes of apneas and hypopneas lasting ten seconds or more by the number of hours of sleep. The higher the index the more serious the condition. An index between 5 and 10 is low, between 10 and 15 is mild to moderate, over 15 is moderately severe, and anything over 30 indicates severe sleep apnea.

Systems and methods according to the disclosure may utilize one or more implanted or external (e.g., non-invasive) sensors to detect signals indicative of one or more physiological biomarkers of the subject. For example, in some aspects, the sensor comprises a heart rate sensor, a blood pressure sensor, an SpO₂ (oxygen saturation) sensor, an electroencephalogram (“EEG”) sensor (e.g., positioned on a patient's scalp or behind a patient's ear). In some aspects, the sensor comprises an electrocardiogram (“EKG”) sensor or an electromyography (“EMG”) sensor. In some aspects, the sensor comprises a microphone or an inertial measurement unit (“IMU”), and the detected signal is indicative of a heart rate or an ictal tachycardia event.

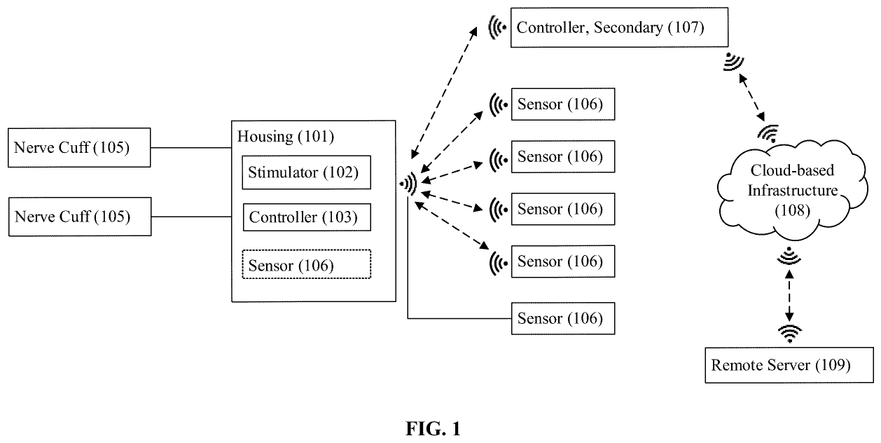

FIG. 1 is a block diagram illustrating an exemplary embodiment of a system for adaptive VNS in accordance with the present disclosure. In this example, a single implanted housing (101) contains a stimulator (102) (e.g., an implantable pulse generator, “IPG”) and a controller (103), wherein the housing (101) is further connected to two nerve cuffs (each comprising one or more electrodes) via a bifurcated electrode lead. The controller (103) in this VNS device is further shown to be in communication with various sensors (106) via a series of wireless or wired connections, and with a remote server (109).

As illustrated by this figure, the implanted housing (101) may fully contain the stimulator (102). In other aspects, the stimulator (102) may be only partially contained within the housing (101). This figure also illustrates the inclusion of a sensor (106) within the housing (101). It is understood that one or more sensors may optionally be included in the implanted housing (101). For example, an IMU or microphone may be integrated into the housing (101) and directly connected to the controller (103) within the housing. In other aspects, the housing (101) may not contain any sensors (e.g., to minimize the size of the implant) and may instead communicate wirelessly or via a wired connection with one or more separately implanted or external sensors. For example, here the controller (103) is communicatively linked with five sensors via wireless connections, and with one sensor via a wired connection. Each of these sensors may comprise any of the various sensor types described herein or otherwise known in the art. In some aspects, each sensor may comprise a dedicated hardware device, or a sensor integrated into another device (e.g., a sensor integrated into a smart watch or other fitness device worn, placed, or strapped to a subject).

This figure further illustrates that the controller (103) integrated into the implanted VNS device may be communicatively linked to an external secondary controller (107). A secondary controller (107) may comprise a dedicated hardware device, or software executed on a computer, tablet, or other electronic device. The secondary controller (107) may in turn be capable of communicating with one or more remote servers (109) via a cloud-based or other network. For example, the secondary controller (107) may communicate with a remote server (109) operated by a clinician programmer, allowing for patient data to be shared with the clinician and/or for the clinician to remotely modify one or more parameters of the VNS device.

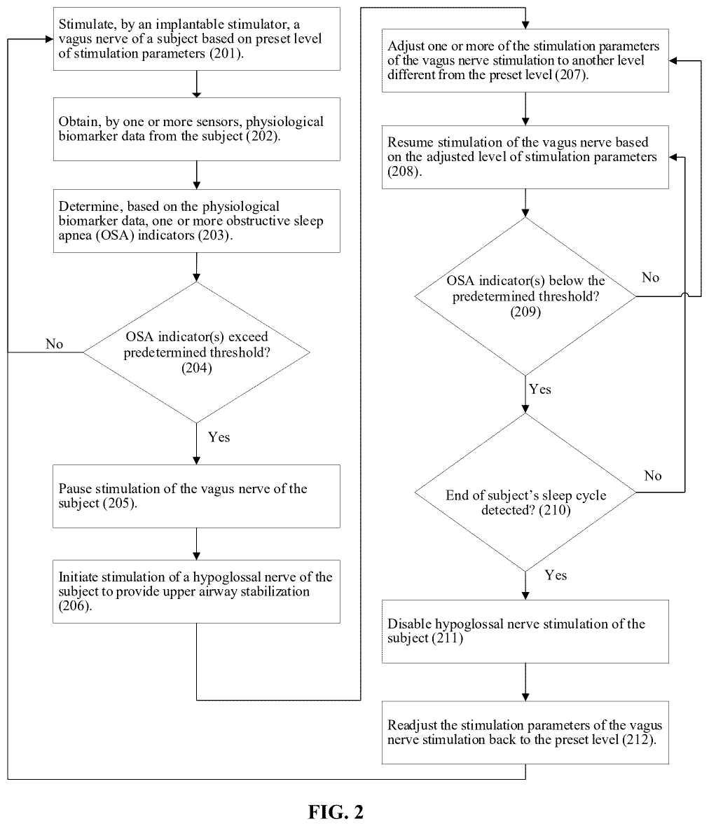

FIG. 2 is a conceptual flow diagram of a process for treating a subject using VNS using an adaptive algorithm according to an exemplary aspect of the disclosure. As illustrated by this figure, treatment may begin with the stimulation of a subject's vagus nerve, by an implantable stimulator, based on preset level of stimulation parameters (201). The device may be implanted in the neck, chest, or anywhere else deemed suitable by a qualified medical professional. Following surgical implantation, a subject may initiate stimulation of their vagus nerve using the VNS device (e.g., the subject may utilize a secondary controller (107) to activate delivery of stimulation to the vagus nerve). Initially, stimulation may be based on clinically evaluated parameters known to be safe and/or effective. Such parameters may be derived from a personalized study of the subject or based on the results of a population study. In either case, while stimulation is being delivered, one or more sensors (106) may be activated in order to detect signals indicative of one or more physiological biomarkers of the subject (e.g., heart rate, blood pressure, oxygen level, gross movements) (201). This signal data may be transmitted to the controller (103) for processing, and the controller (103) may be configured to determine one or more obstructive sleep apnea (OSA) indicators (interchangeably referred to as indicators) including an AHI, respiration rate, ODI, and/or or any marker indicative of the subject experiencing an obstructive sleep apnea, based on the collected signals (203).

If the OSA indicator(s) are determined (204) not to exceed one or more predetermined thresholds (e.g., by remaining stable, increasing but not exceeding the relevant threshold, or decreasing), the controller (103) may continue in this monitoring cycle as shown by FIG. 2. Alternatively, if an increase in one or more OSA indicator(s) such as an AHI, respiration rate, ODI, or a combination of one or more of these indicators, is determined to exceed one or more predetermined thresholds (204), the controller (103) may be configured to proceed with adaptive modulation of the VNS treatment. For example, the controller (103) may pause stimulation of the subject's vagus nerve (205), and initiate stimulation of the subject's hypoglossal nerve (206). The hypoglossal nerve stimulation may be performed to induce upper airway stabilization and patency.

Next, the controller (103) may be configured to adjust one or more of the stimulation parameters of the vagus nerve stimulation to another level different from the preset level (207); and to cause the stimulator (102) to resume stimulation of the subject's vagus nerve (208) using the one or more adjusted parameters (pulse width, frequency, duty cycle, etc.). The adjustment of parameters at this stage illustrates the adaptive nature of the algorithms implemented herein. In some aspects, an adjusted parameter may be set to a value that is ±1, 2, 3, 4, 5, 6, 7, 8, 9, 10, 11, 12, 13, 14, 15, 16, 17, 18, 19, or 20% compared to the value of the preset level, or to a value within a range with endpoints defined by any pair of the foregoing values. At this point, the controller (103) may utilize sensor data collected from the communicatively linked sensors to evaluate whether the adjusted VNS parameters are effective (e.g., by once again evaluating the subject's OSA indicator(s), such as an AHI, respiration rate, ODI, or a combination of one or more of these indicators) (209). In some aspects, the controller (103) may base this determination on a comparison of one or more current OSA indicator(s) in relation to one or more previously recorded OSA indicator(s) (e.g., the subject's AHI, respiration rate, ODI or a combination of one or more of these indicators, prior to initiation of the VNS, or the level of any of the parameters before the immediately preceding round of VNS stimulation). In each case, a rolling average may be utilized for the current and/or previously recorded level.

At this stage, the controller may also be configured to determine whether the subject's sleep cycle has ended (210). For example, the controller (103) may utilize one or more sensor signals indicative of the subject's physical orientation or movement (e.g., obtained from an inertial measurement unit such as an accelerometer, a gyroscope, or a combination thereof) in order to determine whether the subject is awake or asleep. Similarly, the controller (103) may be configured to receive manual user input indicative of the start or end of a sleep cycle (e.g., a user may use an interface of an external controller to indicate that they are awake. In some aspects, the controller (103) may be configured to store one or more preset time schedules indicative of a sleep cycle of the subject (e.g., a subject may be able to manually select start and end time points for a sleep cycle in order to establish a window within stimulation may be administered).

If the controller determines that the subject's OSA indicator(s) exceed one or more predetermined thresholds or ranges (209) (e.g., as compared to the level of the selected OSA indicator(s) prior to initiation of the VNS, or a predetermined level for any of the OSA indicator(s) set by a clinician or the user), the controller (103) may further adjust one or more parameters of the VNS (returning to step 207). At this stage, the controller may also take into account whether the subject's sleep cycle has ended (210), e.g., based on sensor data, manual input from the subject, or a preset schedule, as described above. If the controller determines that one or more OSA indicators are below one or more predetermined thresholds (209) and that the subject's sleep cycle has ended (210), the controller may then disable hypoglossal nerve stimulation (211) and set one or more parameters of the VNS to a predetermined amount or level (e.g., a clinically evaluated amount of value known to be safe and/or effective (212), before resuming the cycle (i.e., by returning to 201).

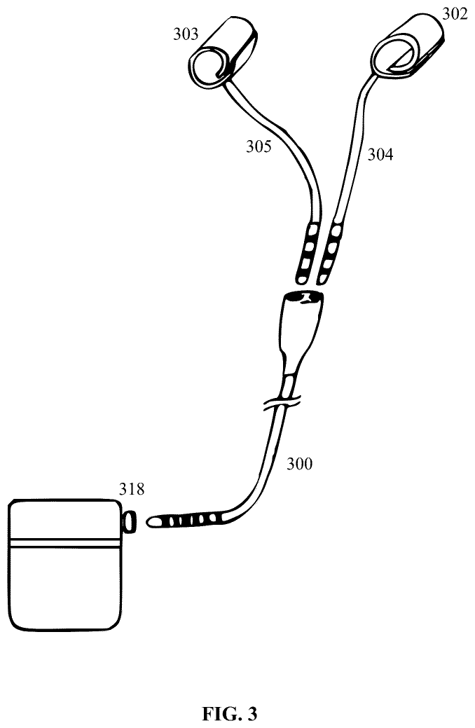

As noted above, in some aspects the stimulator may utilize a lead adaptor configured to couple to two or more leads. Each lead may comprise one or more electrodes that may be used to stimulate different locations on the same nerve, and/or different nerves (e.g., the hypoglossal and/or vagus nerves). An exemplary lead adaptor is shown in FIG. 3, in this case a splitter that allows an IPG header receptacle 318 to couple two different leads (304, 305), each associated with a respective nerve cuff (302, 303).

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

We claim:

1. A system for vagus nerve stimulation, comprising:
a stimulator implanted in a subject and configured to deliver electrical stimulation to a vagus nerve of the subject and to a hypoglossal nerve of the subject; and
one or more sensors, each configured to detect a signal indicative of a level of a physiological biomarker of the subject; and
a controller configured to
determine one or more obstructive sleep apnea (“OSA”) indicators for the subject, based on at least one signal detected by the one or more sensors, wherein the OSA indicators comprise an apnea-hypopnea index (“AHI”), respiration rate, oxygen desaturation index (“ODI”), or a combination thereof; and
control delivery of the electrical stimulation to the vagus nerve of the subject, and delivery of the electrical stimulation to the hypoglossal nerve of the subject, based on the determined OSA indicator(s),
pause stimulation of the vagus nerve and initiate stimulation to the hypoglossal nerve, in response to determining an increase in the one or more OSA indicators, beyond the at least one preset threshold, and
adjust one or more parameters of the electrical stimulation administered to the vagus nerve, prior to resuming the administration of stimulation to the vagus nerve.

2. The system of claim 1, wherein the controller is further configured to pause stimulation of the vagus nerve for a predetermined time in response to determining an increase in the one or more OSA indicators, beyond at least one preset threshold.

3. The system of claim 1, wherein the OSA indicators for the subject comprise two or more of an AHI, respiration rate, and ODI of the subject, and the controller is further configured to initiate stimulation to the hypoglossal nerve of the subject in response to determining an increase in the two or more OSA indicators, beyond the at least one preset threshold for each respective OSA indicator.

4. The system of claim 1, wherein the one or more sensors comprise at least one external sensor.

5. The system of claim 1, wherein the controller is further configured to resume administering stimulation to the vagus nerve after initiating stimulation to the hypoglossal nerve.

6. The system of claim 1, wherein the controller is further configured to adjust a pulse frequency, width, amplitude, and/or duty cycle of the electrical stimulation administered to the vagus nerve, prior to resuming the administration of stimulation to the vagus nerve.

7. The system of claim 1, wherein the controller is further configured to:
a) monitor the one or more OSA indicators after resuming the administration of stimulation to the vagus nerve; and/or
b) determine whether the subject is asleep.

8. The system of claim 7, wherein the controller is further configured to
disable hypoglossal nerve stimulation when (i) the one or more OSA indicators exceed the at least one preset threshold, and (ii) the subject is determined to be awake.

9. The system of claim 7, wherein the controller is further configured to adjust one or more parameters of the electrical stimulation administered to the vagus nerve after disabling the hypoglossal nerve stimulation.

10. The system of claim 6, wherein the one or more parameters of the electrical stimulation comprise a pulse frequency, width, amplitude, and/or duty cycle of the electrical stimulation delivered to the vagus nerve.

11. The system of claim 6, wherein the at least one preset threshold comprises:
a threshold OSA indicator level selected by a clinician;
a threshold OSA indicator level selected by a clinician based on historical data for the OSA indicator obtained from the subject; and/or
a threshold OSA indicator level previously determined to define a tolerable or intolerable OSA indicator level for the subject.

12. The system of claim 11, wherein the at least one preset threshold comprises a plurality of thresholds, each for a different OSA indicator, optionally including a threshold for the AHI, a threshold for the respiration rate, and/or a threshold for the ODI; and
wherein the controller is configured to administer or disable stimulation based on two or more of the plurality of thresholds.

13. The system of claim 1, wherein the one or more sensors comprise a heart rate sensor, a blood pressure sensor, an SpO₂ sensor, an electroencephalogram (“EEG”) sensor, an electrocardiogram (“ECG”) sensor); and/or an electromyography (“EMG”) sensor.

14. The system of claim 1, wherein the stimulator comprises
a) a pulse generator configured to deliver electrical stimulation to a bifurcated electrode lead that comprises a plurality of electrodes placed on a hypoglossal nerve cuff, and a plurality of electrodes placed on a vagus nerve cuff; or
b) a pulse generator configured to deliver electrical stimulation to a lead adaptor configured to couple to two or more leads.

15. The system of claim 1, wherein the controller is further configured to detect the onset of a seizure in the subject, based on at least one signal detected by the one or more sensors, and to control the delivery of the electrical stimulation to the vagus nerve and/or to the hypoglossal nerve of the subject based on the detection of the seizure.

16. The system of claim 15, wherein the controller is further configured to detect ictal tachycardia events experienced by the subject, based on at least one signal detected by the one or more sensors.

17. The system of claim 16, wherein the controller is further configured to detect the onset of a seizure in the subject based on the detection of an ictal tachycardia event experienced by the subject.

18. A method for vagus nerve stimulation (“VNS”), comprising:
a) providing a stimulator implanted in a subject and configured to deliver electrical stimulation to a vagus nerve of the subject, and to a hypoglossal nerve of the subject;
b) detecting, by at least one sensor, a signal indicative of a level of a physiological biomarker of the subject;
c) providing a controller configured to
i) determine one or more obstructive sleep apnea (“OSA”) indicators for the subject based on at least one signal detected by the one or more sensors, wherein the one or more OSA indicators comprise an apnea-hypopnea index (“AHI”), respiration rate, oxygen desaturation index (“ODI”), or a combination thereof; and
ii) control the delivery of the electrical stimulation to the vagus nerve of the subject, and/or to the hypoglossal nerve of the subject, based on the determined OSA indicator(s),
iii) pause stimulation of the vagus nerve and initiate stimulation to the hypoglossal nerve, in response to determining an increase in the one or more OSA indicators, beyond the at least one preset threshold, and
iv) adjust one or more parameters of the electrical stimulation administered to the vagus nerve, prior to resuming the administration of stimulation to the vagus nerve.

19. The method of claim 18, wherein the controller is further configured to pause stimulation of the vagus nerve for a predetermined time in response to determining an increase in the one or more OSA indicators, beyond at least one preset threshold.

20. The method of claim 18, wherein the OSA indicators for the subject comprise two or more of an AHI, respiration rate, and ODI of the subject, and the controller is further configured to initiate stimulation to the hypoglossal nerve of the subject in response to determining an increase in the one or more OSA indicators, beyond the at least one preset threshold.

21. The method of claim 18, wherein:
a) the one or more sensors comprise at least one external sensor;
b) the controller is further configured to resume administering stimulation to the vagus nerve after initiating stimulation to the hypoglossal nerve; and/or
c) the controller is further configured to: i) monitor the one or more OSA indicators, after resuming the administration of stimulation to the vagus nerve; and/or ii) determine whether the subject is asleep.

22. The method of claim 21, wherein the controller is further configured to:
disable hypoglossal nerve stimulation when (i) the one or more OSA indicators exceed the at least one preset threshold, and (ii) the subject is determined to be awake.

23. The method of claim 22, wherein the controller is further configured to adjust one or more parameters of the electrical stimulation administered to the vagus nerve after disabling the hypoglossal nerve stimulation.

24. The method of claim 21, wherein the one or more parameters of the electrical stimulation comprise a pulse frequency, width, amplitude, and/or duty cycle of the electrical stimulation delivered to the vagus nerve.

25. The method of claim 19, wherein the at least one preset threshold comprises:
a threshold OSA indicator level selected by a clinician;
a threshold OSA indicator level selected by a clinician based on historical data for the OSA indicator obtained from the subject; and/or
a threshold OSA indicator level previously determined to define a tolerable or intolerable OSA indicator level for the subject.
